(19) United States
(12) Patent Application Publication
(10) Pub. No.: US 2010/0305163 A1
(43) Pub. Date: Dec. 2, 2010

(54) PHARMACEUTICAL FORMULATIONS COMPRISING NSAID AND PROTON PUMP INHIBITOR DRUGS

(76) Inventors: Pramod Dattatray Yedurkar, Mumbai (IN); Pramod Kharwade, Chhindwara (IN); Narayanan Badri Vishwanathan, Chennai (IN); Sandeep Mhetre, Sangli (IN); Vinay Kumar Chiluka, Adilabad (IN); Siva Satya Krishna Babu Pechetti, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/782,964

(22) Filed: May 19, 2010

Related U.S. Application Data
(60) Provisional application No. 61/291,233, filed on Dec. 30, 2009.

(30) Foreign Application Priority Data
May 20, 2009 (IN) 1158/CHE/2009
Mar. 25, 2010 (IN) 806/CHE/2010

(51) Int. Cl. A61K 31/4439; A61P 19/02; A61P 1/04; A61P 29/00

(52) U.S. Cl. 514/338

(57) ABSTRACT

Aspects of the invention relate to pharmaceutical formulations comprising an NSAID and acid reducer drug for therapeutic purposes, and methods of preparing the same. Further aspects of the invention relate to fixed dose pharmaceutical formulations comprising naproxen, or pharmaceutically acceptable salts thereof, and esomeprazole, or pharmaceutically acceptable salts thereof.

### INTRODUCTION

Aspects of the present invention relate to pharmaceutical formulations comprising an NSAID and a proton pump inhibitor, for therapeutic purposes, and methods of preparing the same. Further aspects of the present invention relate to fixed dose pharmaceutical formulations comprising naproxen, or pharmaceutically acceptable salts thereof, and esomeprazole, or pharmaceutically acceptable salts thereof.

Naproxen is a propionic acid derivative in the arylacetic acid group of nonsteroidal anti-inflammatory drugs (NSAID). A chemical name for naproxen is (S)-6-methoxy-α-methyl-2-naphthaleneacetic acid. It has been available for many years and has been found to be acceptably non-toxic by many regulatory agencies. Naproxen is used as either its free acid form or its sodium salt, naproxen sodium. The compounds are represented by structure (1), where R═COOH for naproxen and R═COONa for naproxen sodium.

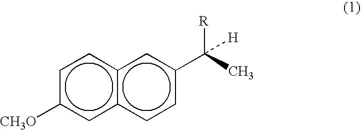

Naproxen has a molecular weight of 230.26 and a molecular formula C₁₄H₁₄O₃. It is an odorless, white to off-white crystalline substance. It is lipid-soluble, practically insoluble in water at low pH and freely soluble in water at high pH. The octanol/water partition coefficient of naproxen at pH 7.4 is 1.6 to 1.8. NAPROSYN™ (naproxen tablets) are available from Hoffman-La Roche as yellow tablets containing 250 mg of naproxen, pink tablets containing 375 mg of naproxen and yellow tablets containing 500 mg of naproxen for oral administration. EC-NAPROSYN™ (naproxen delayed-release tablets) are available as enteric-coated white tablets containing 375 mg and 500 mg of naproxen for oral administration. ANAPROX™ (naproxen sodium tablets) is available from Hoffman-La Roche as blue tablets containing 275 mg of naproxen sodium and ANAPROX™ DS (naproxen sodium tablets) is available as dark blue tablets containing 550 mg of naproxen sodium for oral administration. NAPROSYN™ (naproxen suspension) is available as a light orange-colored opaque oral suspension containing 125 mg/5 mL of naproxen, in a vehicle.

Esomeprazole is a proton pump inhibitor that reduces gastric acid secretion through inhibition of H/K⁺-ATPase enzyme in gastric parietal cells. It was developed and is marketed by AstraZeneca in products sold as NEXIUM™ which is used in the treatment of dyspepsia, peptic ulcer disease, gastro-esophageal reflux disease and Zollinger-Ellison syndrome. Esomeprazole is the S-enantiomer of omeprazole (marketed as PRILOSECT™), having improved efficacy over the racemic mixture of omeprazole. It has a molecular formula C₁₇H₁₈N₃O₃S and is represented by structure (2).

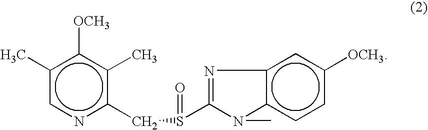

NEXIUM™ products are supplied in delayed-release capsules and in packets for a delayed-release oral suspension. Each delayed-release capsule contains 20 mg or 40 mg of esomeprazole (as esomeprazole magnesium trihydrate) in the form of enteric-coated granules. Each packet of NEXIUM™ For Delayed-Release Oral Suspension contains 10 mg, 20 mg, or 40 mg of esomeprazole, in the form of the same enteric-coated granules used in NEXIUM™ Delayed-Release Capsules, and also inactive granules. The esomeprazole granules and inactive granules are constituted with water to form a suspension and are given by oral, nasogastric or gastric administration.

NSAIDs, including naproxen, are among the most commonly prescribed and used drugs world-wide. Despite the therapeutic benefits of NSAIDs, their use is frequently limited by an increased risk of gastrointestinal side-effects, mainly upper gastrointestinal side-effects like peptic ulceration and dyspeptic symptoms. It appears that a major factor contributing to the development of these lesions is the presence of acid in the stomach and upper small intestine of patients. Proton pump inhibitors such as omeprazole have been shown to be able to prevent gastric and duodenal erosions in healthy volunteers during treatment with NSAIDs. Clinical studies have shown, that omeprazole heals gastric as well as duodenal ulcers as quickly and effectively in patients on continuous NSAID treatment as in non-NSAID users.

Recognizing the potential benefits of proton pump inhibitors for the prevention of NSAID-induced gastroduodenal damage, there are strategies disclosed in the art for combining the two types of active agents for therapeutic purposes, other than by concomitant administration. U.S. Pat. No. 6,365,184 suggested a means of delivery that would expose the gastrointestinal tract to NSAIDs prior to onset of PPI activity. U.S. Pat. No. 6,926,907 discloses pharmaceutical formulations that provide coordinated release of an acid inhibitor and an NSAID.

There remains a need for alternative pharmaceutical formulations comprising naproxen and esomeprazole in order to reduce the risk of gastrointestinal side effects in people taking naproxen for pain relief and for other conditions, and thereby improve patient compliance.

### SUMMARY

In aspects, the present invention relates to pharmaceutical formulations comprising a proton pump inhibitor and a non-steroidal anti-inflammatory drug.

Embodiments of the present invention relate to pharmaceutical formulations comprising naproxen and esomeprazole for therapeutic purposes, and methods of preparing the formulations.

In embodiments, the invention includes pharmaceutical formulations comprising naproxen and esomeprazole, wherein at least a portion of naproxen is released when the formulations are immersed into aqueous fluids having pH values below about 3.5.

In embodiments, the invention includes pharmaceutical formulations comprising naproxen and esomeprazole, wherein a portion of naproxen is released when the formulations are immersed into aqueous fluids having pH values less than about 3.5, and the remaining portion of naproxen is released when the formulations are immersed into aqueous fluids having pH values about 3.5 or higher.

In embodiments, the invention includes pharmaceutical formulations comprising naproxen and esomeprazole, wherein at least a portion of naproxen and at least a portion of esomeprazole are released when the formulations are immersed into aqueous fluids having pH values less than about 3.5.

In embodiments, the invention includes pharmaceutical formulations comprising naproxen and esomeprazole, wherein a portion of naproxen and at least a portion of esomeprazole are released when the formulations are immersed into aqueous fluids having pH values less than about 3.5, and a remaining portion of naproxen is released when the formulations are immersed into aqueous fluids having pH values about 3.5 or higher.

In embodiments, the invention includes pharmaceutical formulations comprising naproxen and esomeprazole, wherein at least about 5% of naproxen is released when the formulations are immersed into aqueous fluids having pH values less than about 3.5, and a remaining portion of naproxen is released when the formulations are immersed into aqueous fluids having pH values about 3.5 or higher.

In an aspect, the invention includes methods of preparing pharmaceutical formulations of the present invention.

In an aspect, the invention includes methods of treating patients suffering from pain, inflammation, and/or other conditions using pharmaceutical formulations of the present invention. In a further aspect, the invention includes methods of reducing the risk of gastrointestinal side effects in patients taking naproxen for pain relief and for other conditions using pharmaceutical formulations of the present invention.

### DETAILED DESCRIPTION

Aspects of the present invention relate to pharmaceutical formulations comprising a proton pump inhibitor and a non-steroidal anti-inflammatory drug.

Non-steroidal anti-inflammatory drugs include any of aspirin, ibuprofen, naproxen, flurbiprofen, ketoprofen, oxaprozin, etodolac, indomethacin, ketorolac, lornoxicam, nabumetone, diclofenac, and COX-2 inhibitors such as celecoxib, rofecoxib, meloxicam, piroxicam, valdecoxib, parecoxib and etoricoxib. Salts of the drug compounds also can be used herein.

Proton pump inhibitors include any of drugs such as omeprazole, esomeprazole, pantoprazole, lansoprazole, dexlansoprazole, rabeprazole, and other “acid inhibitors” such as H2 blockers including cimetidine, ranitidine, ebrotidine, pabutidine, lafutidine, loxtidine, and famotidine. Salts of the drug compounds also can be used herein.

For simplification, the discussion herein will pertain primarily to formulations comprising combinations of: the non-steroidal anti-inflammatory drug naproxen, or a salt thereof; and the proton pump inhibitor drug esomprazole, or a salt thereof such as the magnesium salt; but those skilled in the art will recognize that other drugs having similar therapeutic effects can be substituted therefor.

Embodiments of the present invention relate to pharmaceutical formulations comprising naproxen and esomeprazole. Further embodiments of the present invention relate to fixed dose pharmaceutical formulations comprising naproxen, or pharmaceutically acceptable salts thereof, and esomeprazole, or pharmaceutically acceptable salts thereof, for therapeutic purposes, and methods of preparing the same.

As used herein the term “naproxen” includes the compound naproxen, pharmaceutically acceptable salts of naproxen, isomers, solvates and hydrates thereof, and any polymorphic or amorphous forms or combinations thereof. Naproxen is used in the present invention in the range of about 50 to 1500 mg, or about 100 to 1250 mg, or about 350 to 800 mg, or about 275 to 550 mg, or about 250 mg to 500 mg, per dosage unit.

As used herein the term “esomeprazole” includes the compound esomeprazole, pharmaceutically acceptable salts of esomeprazole, solvates and hydrates thereof, and any polymorphic or amorphous form or combinations thereof. Esomeprazole is used in the present invention in the range of about 5 to 100 mg, or about 10 to 60 mg, or about 20 to 40 mg, per dosage unit.

As used herein, the terms “pharmaceutically acceptable salt,” or “salt,” include salts prepared using inorganic acids or bases, and organic acids or bases. Examples include metal salts such as aluminum, calcium, lithium, magnesium, potassium, sodium, and zinc salts. Examples of organic bases include N,N′-dibenzylethylenediamine, chloroprocaine, choline, diethanolamine, ethylenediamine, meglumaine (N-methylglucamine), lysine, and procaine. Examples of inorganic acids are hydrochloric, hydrobromic, hydroiodic, sulfuric, and phosphoric acids. Appropriate organic acids include, for example, aliphatic, aromatic, carboxylic and sulfonic classes of organic acids, some examples of which are formic, acetic, propionic, succinic, glycolic, glucoronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic acids.

The foregoing lists are not intended to be exhaustive, and many other substances can be used.

In embodiments, the invention includes pharmaceutical formulations comprising naproxen and esomeprazole, wherein at least a portion of naproxen is released when the pH of a surrounding medium is below about 3.5.

In embodiments, the invention includes pharmaceutical formulations comprising naproxen and esomeprazole, wherein a portion of naproxen is released when the pH of a surrounding medium is below about 3.5, and the remaining portion of naproxen is released when the pH of a surrounding medium is 3.5 or higher.

Pharmaceutical formulations according to the present invention can be presented in forms such as tablets, multilayered tablets, capsules, granules, spheroids, beads, pellets, mini-tablets, powders, sachets, gels, dispersions, solutions, and suspensions.

As used herein the term “particulate” includes particles such as granules, spheroids, beads, pellets, and mini-tablets.

As used herein the term “at least a portion of naproxen” means at least about 5%, or at least about 10%, or at least about 20%, or at least about 50%, of naproxen contained in a formulation.

The term “about” is related to a pH, ranging from 0 to 3.5.

In embodiments, the invention includes pharmaceutical formulations comprising naproxen and esomeprazole, wherein at least a portion of naproxen and at least a portion of esomeprazole are released when the pH of a surrounding medium is below about 3.5.

In embodiments, the invention includes pharmaceutical formulations comprising naproxen and esomeprazole, wherein a portion of naproxen and at least a portion of esomeprazole are released when the pH of a surrounding medium is below about 3.5, and a remaining portion of naproxen is released when the pH of a surrounding medium is 3.5 or higher.

In embodiments, the invention includes pharmaceutical formulations comprising naproxen and esomeprazole, wherein at least a portion of naproxen and a portion of esomeprazole are released when the pH of a surrounding medium is below about 3.5, and a remaining portion of esomeprazole is released when the pH of a surrounding medium is 3.5 or higher.

As used herein the term “at least a portion of esomeprazole” means at least about 50%, or at least about 70%, or at least about 90%, or about 100%, of esomeprazole contained in a formulation.

In embodiments, the invention includes pharmaceutical formulations comprising naproxen and esomeprazole, wherein at least a portion of naproxen begins to be released from the pharmaceutical formulation immediately following administration, or within about 0.5 hour, or within about 1 hour, or within about 2 hours, following administration.

In an embodiment, the invention includes pharmaceutical formulations comprising naproxen and esomeprazole, wherein at least a portion of esomeprazole begins to be released from the pharmaceutical formulation immediately following administration, or within about 0.5 hour, or within about 1 hour, or within about 2 hours, following administration.

Pharmaceutical formulations of embodiments of the present invention are advantageous as they provide drug release in such a way that they provide a rapid onset of action, due to the release of at least a portion of naproxen when the pH of the surrounding medium is below about 3.5. Also, they provide release of at least a portion of esomeprazole along with at least a portion of naproxen, when the pH of the surrounding medium is below about 3.5, which elevates gastric pH before the release of the remaining portion of naproxen, occurring when the pH of the surrounding medium is 3.5 or higher. This tends to alleviate some of the deleterious effects of the naproxen on the gastro-duodenal mucosa.

Release of drugs from their formulations in the body can be simulated using in vitro methods such as those described in Test 711 “Dissolution” of United States Pharmacopeia 29, United States Pharmacopeial Convention, Rockville, Md., 2005 (“USP”). Release profiles are determined by immersing a dosage form in an aqueous fluid and measuring the drug concentration in the fluid at various elapsed times. The USP gives compositions for various aqueous fluids that correspond to physiologic fluids, in test methods and in monographs for individual drug substances. Drug concentrations in dissolution fluids can be determined using techniques such as high performance liquid chromatography.

In embodiments, the invention includes pharmaceutical formulations comprising naproxen and esomeprazole, wherein the formulations are in monolithic form or multiparticulate form. The present invention includes oral fixed unit dose formulations such as enteric coated tablets, multilayered tablets, multiple unit tableted dosage forms, capsules filled with enteric coated pellets, etc.

In embodiments, the invention includes pharmaceutical formulations comprising naproxen and esomeprazole, wherein the formulations are bi-layer tablets having an outer layer comprising at least a portion of the contained naproxen and at least a portion of the contained esomeprazole, and an inner core which comprises the remaining portion of naproxen.

The layers containing an active ingredient can be referred to as “active layers” and a layer that generally does not contain an active substance can be referred to as a “barrier layer” or a “support layer.” Multi-layered tablets of the present invention can be prepared as two-layer tablets, three-layer tablets, or in greater numbers of layers if desired. In embodiments, at least one layer will contain an active substance to be released from the tablet and least one layer will be a barrier or support layer with respect to the active substance-containing layer or layers.

In embodiments, the invention includes pharmaceutical formulations comprising naproxen and esomeprazole, wherein said formulations are multi-layer tablets having an active layer comprising at least a portion of the contained naproxen and at least a portion of the contained esomeprazole, an inner core comprising the remaining portion of naproxen, and a polymeric barrier layer surrounding the inner core that does not dissolve unless the surrounding medium has a pH of at least about 3.5, or at a pH of at least about 4, or at a pH of at least about 5.

In embodiments, the invention includes multi-layer tablets comprising: (a) an active layer comprising at least a portion of naproxen; (b) a second active layer comprising at least a portion of esomeprazole; (c) an inner core comprising the remaining portion of naproxen; and (d) and a polymeric barrier layer surrounding the inner core that does not dissolve unless the surrounding medium has a pH of at least 3.5; wherein the active layer (a) is formed over polymeric barrier layer (d). In another embodiment, the active layer (b) is formed over polymeric barrier layer (d).

Multi-layered tablets of the invention can be prepared using methods known to those having skill in the art.

In embodiments, pharmaceutical formulations of the invention optionally have one or more coatings which are functional or non-functional. Functional coatings include controlled release coatings; and non-functional coatings include seal coatings and elegant coatings.

In embodiments, the invention includes pharmaceutical formulations comprising naproxen and esomeprazole, wherein the formulations are in multiparticulate form.

In embodiments, the invention includes pharmaceutical formulations comprising multiparticulates, wherein each particle comprises naproxen and/or esomeprazole, optionally together with one or more pharmaceutically acceptable excipients.

In embodiments, multiparticulates comprising naproxen are composed of a core comprising naproxen and optionally one or more pharmaceutically acceptable excipients.

In embodiments, multiparticulates comprising naproxen are uncoated. In other embodiments, multiparticulates comprising naproxen include one or more coatings over a core.

In embodiments, multiparticulates comprising esomeprazole are composed of a core comprising esomeprazole, and optionally one or more pharmaceutically acceptable excipients.

In embodiments, multiparticulates comprising esomeprazole are uncoated. In other embodiments, multiparticulates comprising esomeprazole include one or more coatings over a core.

In embodiments, multiparticulates have a non-functional seal coating, a functional coating, or both.

In embodiments, an initial seal coating can be applied directly onto a core.

In embodiments, multiparticulates further contain a coating layer comprising one or more enteric polymers, over an initial seal coating, if present, or directly over an uncoated multiparticulate naproxen core, to provide control of drug release.

In embodiments, an enteric coating may be applied to esomeprazole-containing multiparticulates.

In embodiments, enteric-coated multiparticulates are further coated with a final seal coating.

In embodiments, any one or all of the coating formulations optionally contain naproxen, esomeprazole, or a combination thereof.

In embodiments, multiparticulates comprising naproxen comprise a non-pariel core, such as inert sugar or similar substances, unto which naproxen is coated, optionally together with one or more pharmaceutically acceptable excipients, using any techniques such as powder layering, solution spraying, or suspension spraying.

In embodiments, multiparticulates comprising esomeprazole comprise a non-pariel core such as inert sugar or similar substances, onto which esomeprazole is coated, optionally together with one or more pharmaceutically acceptable excipients, using any technique such as powder layering, solution spraying, or suspension spraying.

In embodiments, the formulations of the invention are composed of naproxen-loaded non-pariel cores, having a coating comprising one or more enteric polymers.

In embodiments, the invention includes pharmaceutical formulations comprising multiparticulates, comprising naproxen in a drug-containing core and a coating comprising one or more enteric polymers, and optionally having one or more further coatings.

In embodiments, the invention includes pharmaceutical formulations comprising multiparticulates, comprising esomeprazole in a drug-containing core and a coating comprising one or more enteric polymers, and optionally having one or more further coatings.

In embodiments, multiparticulates comprising naproxen contain a non-functional seal coating, a functional coating, or both.

In embodiments, multiparticulates comprising esomeprazole contain a non-functional seal coating, a functional coating, or both.

In embodiments, an initial seal coating is applied directly to drug-containing cores.

In embodiments, enteric-coated multiparticulates are further coated with a final seal coating.

In embodiments, the invention includes pharmaceutical formulations comprising:

(a) a first set of multiparticulates comprising at least a portion of naproxen, wherein naproxen is released when the pH of a surrounding medium is below about 3.5; (b) a second set of multiparticulates comprising the remaining portion of naproxen, wherein naproxen is released when the pH of a surrounding medium is 3.5 or higher; and (c) a third set of multiparticulates comprising at least a portion of esomeprazole, wherein esomeprazole is released when the pH of a surrounding medium is below about 3.5.

In embodiments, the invention includes pharmaceutical formulations comprising:

(a) a first set of multiparticulates comprising at least a portion of naproxen, wherein naproxen is released when the pH of a surrounding medium is below about 3.5; (b) a second set of multiparticulates comprising the remaining portion of naproxen, wherein naproxen is released when the pH of a surrounding medium is 3.5 or higher; and (c) a third set of multiparticulates comprising esomeprazole, wherein esomeprazole is released when the pH of a surrounding medium is below about 3.5.

The multiparticulate formulations of the invention can be prepared using techniques described herein, as well as other methods known to those having skill in the art.

In embodiments, multiparticulates comprising naproxen and esomeprazole can be combined with a pharmaceutically acceptable carrier, and optionally other excipients, and compounded to form pharmaceutical formulations, i.e., can be compressed into tablets or placed into suitable capsule shells, using techniques known to those having skill in the art.

In embodiments, the invention includes pharmaceutical formulations comprising naproxen and esomeprazole, wherein said formulations include an enteric coating layer which is deliberately formed such that the enteric coating is a partial or thin, “leaky,” or permeable enteric coating. These enteric-coated formulations are prepared such that upon contacting gastric fluid, either in vivo or in vitro, the leaky enteric coating layer allows at least a portion of the active ingredient to be released from the pharmaceutical formulation when the pH of a surrounding medium is below about 3.5. This release begins immediately following administration, or within about 0.5 hour, or within about 1 hour, or within about 2 hours, following administration. The enteric material composition may be made leaky by incorporating pore forming materials that allow gastric fluid to penetrate into the formulation and drug to be released from the formulation when the pH of a surrounding medium is below about 3.5.

In embodiments, the invention includes pharmaceutical formulations comprising naproxen, esomeprazole and a leaky enteric coating layer.

In embodiments, the invention includes pharmaceutical formulations comprising naproxen, esomeprazole and a leaky enteric coating layer from which at least a portion of naproxen in the formulation is released when the pH of a surrounding medium is below about 3.5 and remaining portion of naproxen in the formulation is released when the pH of a surrounding medium is 3.5 or higher.

In embodiments, the invention includes pharmaceutical formulations comprising at least a portion of naproxen in the formulation is released when the pH of a surrounding medium is below about 3.5, such as about 2.5 to 3.5, and a remaining portion of naproxen in the formulation is released when the pH of a surrounding medium is 3.5 or higher.

In embodiments, the invention includes pharmaceutical formulations comprising naproxen, esomeprazole, and an enteric coating layer comprising one or more pore forming agents.

In embodiments, the invention includes pharmaceutical formulations comprising an inner core comprising naproxen and an enteric coating layer on the core comprising one or more pore forming agents.

In embodiments, the invention includes pharmaceutical formulations comprising naproxen substantially homogeneously admixed with at least one enteric material comprising a pore forming agent.

In embodiments, the present invention includes pharmaceutical formulations comprising naproxen, esomeprazole, and an enteric coating layer comprising one or more pore forming agents, wherein the amount of pore forming agent is from about 0-400% by weight of enteric polymer, or from about 5-200% by weight of enteric polymer, or from about 10-100% or from about 15-50% by weight of enteric polymer.

One or more pore forming agents that can be used in the present invention include hydrophilic, hydrophobic, or a combination of both hydrophilic and hydrophobic, substances. Examples of pore forming agents include, without limitation: salts such as sodium chloride, potassium chloride, and magnesium salts; sugars, such as lactose, sucrose, sorbitol, and mannitol; hydroxylated compounds, including polyvinyl alcohols and glycols, such as polyethylene glycol and propylene glycol; cellulose-derived materials, such as hydroxypropyl celluloses and hydroxy propyl methycelluloses; methacrylic acid copolymers; miscellaneous materials such as croscarmellose sodium, crospovidone sodium starch glycolate, talc, silicon dioxide, polyvinylpyrrolidones, gelling agents such as carbopol, and xanthan gum; and any mixtures thereof.

An inner core comprising naproxen or esomeprazole may be prepared using optional pharmaceutically acceptable excipients such as binders, fillers, disintegrants, lubricants, etc., by methods known to those having skill in the art such as low- or high-shear granulation, wet granulation, slugging, fluidized-bed granulation, and extrusion-spheronization. The manufactured inner core can be used for further processing, e.g., can be provided with an enteric coating material and can further be coated with active ingredient layers comprising naproxen or esomeprazole, optionally using separating functional or non-functional coating layers.

The active ingredient layers comprising naproxen or esomeprazole, together with optional pharmaceutically acceptable excipients such as fillers, binders, plasticizers, anti-tacking and anti-static agents, etc., can be applied to an inner core by coating or layering procedures in suitable equipment such as coating pans, coating granulators, or in a fluidized bed apparatus, using water and/or organic solvents for the coating process.

An inner core or active layer comprising esomeprazole may also contain alkaline, pharmaceutically acceptable substances. Such substances include, but are not limited to, substances such as the sodium, potassium, calcium, magnesium and aluminum salts of phosphoric acid, carbonic acid, citric acid, and other suitable weak inorganic or organic acids, aluminum hydroxide, sodium bicarbonate, substances frequently used in antacid preparations such as aluminum, calcium and magnesium hydroxides; magnesium oxide or composite substances, such as Al₂O₃.6MgO.CO₂.12H₂O, (Mg₆Al₂(OH)₁₆CO₃.4H₂O), MgO.Al₂O₃.2SiO₂.nH₂O and similar compounds, organic pH-buffering substances such as trihydroxymethyl aminomethane, basic amino acids and their salts, and other pharmaceutically acceptable pH-buffering substances.

In embodiments, the present invention includes pharmaceutical formulations comprising naproxen, esomeprazole and at least one alkaline substance, wherein the amount of alkaline substance is from about 10 to about 1800% by weight of esomeprazole, or from about 25 to about 1500% by weight of esomeprazole, or from about 50 to about 1200% by weight of esomeprazole, or from about 100 to about 900% by weight of esomeprazole.

Enteric polymers that can be used in the present invention include, without limitation thereto, cellulose acetate phthalates, hydroxypropyl methylcellulose phthalates, polyvinyl acetate phthalates, hydroxypropyl methylcellulose acetate succinates, cellulose acetate trimellitates, hydroxypropyl methylcellulose succinates, cellulose acetate succinates, cellulose acetate hexahydrophthalates, cellulose propionate phthalates, copolymers of methylmethacrylic acid and methyl methacrylate, copolymers of methyl acrylate, methylmethacrylate and methacrylic acid, copolymers of methylvinyl ether and maleic anhydride (Gantrez™ ES products), ethyl methyacrylate-methylmethacrylate-chlorotrimethylammonium ethyl acrylate copolymers, natural resins such as zein, shellac and copal collophorium, carboxymethyl ethylcellulose, co-polymerized methacrylic acid/methacrylic acid methyl esters such as, for instance, materials known under the trade name EUDRAGIT® L12.5, L100, S12.5, and S100, and several commercially available enteric dispersion systems (e.g., EUDRAGITt®L30D55, FS30D, L100-55, and S100, all sold by Evonik Industries, Germany), KOLLICOAT® MAE30D and 30DP (BASF), ESTACRYL® 30D (Eastman Chemical), AQUATERIC® and AQUACOAT® CPD30 (FMC), and any mixtures thereof.

Enteric coating layers are applied onto an inner core, or onto an inner core covered with a seal coating layer, using suitable coating techniques. The enteric coating layer material may be dispersed or dissolved in either water or in a suitable organic solvent. One or more enteric coating layers, separately or in combination, can be used.

In certain embodiments, pharmaceutical formulations of the present invention optionally include additives additional to the active agents, which include without limitation diluents, binders, disintegrants, surfactants, and other additives that are commonly used in solid dosage form preparations.

### Diluents

Various useful fillers or diluents include but are not limited to starches, lactose, cellulose derivatives, confectioner's sugar and the like. Different grades of lactose include but are not limited to lactose monohydrate, lactose DT (direct tableting), lactose anhydrous, FLOWLAC™ (available from Meggle Products), PHARMATOSE™ (available from DMV) and others. Different starches include but are not limited to maize starch, potato starch, rice starch, wheat starch, pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products), and others. Different cellulose materials that can be used include crystalline celluloses and powdered celluloses. Examples of crystalline cellulose products include but are not limited to CEOLUS™ KG801, AVICEL™ PH101, PH102, PH301, PH302, PH-F20, PH-112, microcrystalline cellulose 114, and microcrystalline cellulose 112. Other useful diluents include but are not limited to carmellose, sugar alcohols such as mannitol (PEARLITOL™ SD200), sorbitol and xylitol, calcium carbonate, magnesium carbonate, dibasic calcium phosphate, and tribasic calcium phosphate.

### Binders

Various useful binders include but are not limited to hydroxypropylcelluloses, also called HPC (KLUCEL™ LF, or EXF) and useful in various grades, hydroxypropyl methylcelluloses, also called hypromelloses or HPMC (METHOCEL™) and useful in various grades, polyvinylpyrrolidones or povidones (such as grades PVP-K25, PVP-K29, PVP-K30, and PVP-K90), PLASDONE™ S 630 (copovidone), powdered acacia, gelatin, guar gum, carbomers (CARBOPOL™), methylcelluloses, polymethacrylates, and starches.

### Disintegrants

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, KOLLIDON™ CL [manufactured by BASF (Germany)], POLYPLASDONE™ XL, XI-10, and INF-10 manufactured by ISP Inc. (USA), and low-substituted hydroxypropylcelluloses. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Surface-Active Agents

Useful surface-active agents include non-ionic, cationic and anionic surface-active agents. Useful non-ionic surface-active agents include ethylene glycol stearates, propylene glycol stearates, diethylene glycol stearates, glycerol stearates, sorbitan esters (SPAN™ products) and polyhydroxyethylenically treated sorbitan esters (TWEEN™ products), aliphatic alcohols and PEG ethers, phenol and PEG ethers. Useful cationic surface-active agents include quaternary ammonium salts (e.g., cetyltrimethylammonium bromide) and amine salts (e.g., octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as for example soybean lecithin and egg yolk.

### Lubricants

An effective amount of any pharmaceutically acceptable tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants

One or more glidant materials, which improve the flow properties of powder blends and minimize dosage form weight variations can be used. Useful glidants include but are not limited to silicone dioxide, talc, and combinations thereof.

### Coloring Agents

Coloring agents can be used to color code the formulations, for example, to indicate the type and dosage of the therapeutic agent therein. Suitable coloring agents include, without limitation, natural and/or artificial compounds such as FD&C coloring agents, natural juice concentrates, pigments such as titanium oxide, iron oxides, silicon dioxide, zinc oxide, combinations thereof, and the like.

### Solvents

Various solvents can be used in the processes for preparation of pharmaceutical formulations of the present invention, including but not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsuiphoxide, N,N-dimethylformamide, tetrahydrofuran, and any mixtures thereof.

In certain embodiments, pharmaceutical formulations of the present invention optionally include one or more polymers additional to the active agents, additives and enteric polymers that are commonly used in solid dosage form preparations.

One or more polymers that can be used in the present invention include hydrophilic, hydrophobic, and lipophilic substances, and combinations thereof. Examples of polymers that can be used in present invention include, without limitation thereto, cellulose ethers, e.g., hydroxypropyl methylcelluloses or hypromelloses (HPMC), hydroxypropylcelluloses (HPC), hydroxyethylcelluloses, ethylcelluloses and carboxymethylcellulose sodium, polymers of vinylpyrrolidone, including homopolymers (povidones) and cross-linked polyvinylpyrrolidones, carboxymethylstarches, polyethylene glycols, polyoxyethylenes, poloxamers (polyoxyethylene-polyoxypropylene copolymers), polyvinylalcohols, glucanes (glucans), carrageenans, scleroglucanes (scieroglucans), mannans, galactomannans, gellans, alginic acid and derivatives (e.g., sodium or calcium alginate, propylene glycol alginate), polyaminoacids (e.g. gelatin), methyl vinyl ether/maleic anhydride copolymers, polysaccharides (e.g. carageenan, guar gum, xanthan gum, tragacanth and ceratonia), alpha, beta or gamma cyclodextrins, and dextrin derivatives (e.g. dextrin), polymethacrylates (e.g. copolymers of acrylic and methacrylic acid esters containing quaternary ammonium groups); acrylic acid polymers (e.g., carbomers); shellac, and derivatives thereof, cellulose acetate, cellulose butyrate, cellulose diacetate, cellulose triacetate, cellulose propionate, cellulose acetate butyrate, and other acetylated cellulose derivatives, etc.

Examples of lipophilic substances that can be used in the present invention include, without limitation thereto, waxes (e.g., carnauba wax, microcrystalline wax, beeswax, polyethoxylated beeswax); natural fats (coconut, soya, cocoa) including modified forms such as totally or partially hydrogenated, hydrogenated castor oil, hydrogenated vegetable oil, and fatty acid derivatives such as mono-, bi- and tri-substituted glycerides, phospholipids, glycerophospholipids, glyceryl palmitostearate, glyceryl behenate, glyceryl monostearate, diethylene glycol palmitostearate, polyethylene glycol stearate, polyethylene glycol palmitostearate, polyoxyethylene-glycol palmitostearate, glyceryl monopalmitostearate, cetyl palmitate, fatty alcohols associated with polyethoxylate fatty alcohols, cetyl alcohol, stearic acid, saturated or unsaturated fatty acids and their hydrogenated derivatives, lecithin, cephalins, chitosan and derivatives thereof, sphingolipids, sterols such as cholesterol and its substituted derivatives, etc.

Coating materials used for film coating include the polymers described above. Useful additives for coating include but are not limited to plasticizers, antiadherents, opacifiers, solvents, and optionally colorants, lubricants, pigments, antifoam agents, and polishing agents.

Various useful plasticizers include but are not limited to substances such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. An opacifier like titianium dioxide may also be present in an amount ranging from about 10% (w/w) to about 20% (w/w) based on the total weight of the coating.

Antiadhesives are frequently used in the film coating process to avoid sticking effects during film formation and drying. An example of a useful antiadhesive for this purpose is talc. The antiadhesive is frequently present in the film coating in an amount of about 5% (w/w) to about 15% (w/w) based upon the total weight of the coating.

When coloured tablets are desired, the colour is normally applied in the coating. Consequently, colouring agents and pigments may be present in the film coating. Various colouring agents include but are not limited to iron oxides, which can be red, yellow, black or blends thereof.

Suitable polishing agents include polyethylene glycols of differing molecular weights and mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In some embodiments, polyethylene glycols having molecular weights of 3,000-20,000 are employed.

As alternatives to the above coating ingredients, sometimes pre-formulated coating products such as OPADRY™ (supplied by Colorcon) will be used. Opadry formulations generally comprise polymer, plasticizer and, if desired, pigment in a dry concentrate that requires only dispersion in a liquid prior to use. Opadry formulas produce attractive, elegant coatings on a variety of tablet cores and can be used in both aqueous and organic coating procedures.

In an embodiment, the invention includes methods of preparing pharmaceutical formulations of the present invention.

Equipment suitable for processing the pharmaceutical formulations of the present invention include rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans; tray dryers, fluid bed dryers, rotary cone vacuum dryers, and the like, multimills, fluid energy mills, ball mills, colloid mills, roller mills, hammer mills, and the like, and different sieves. All sieves that are used for processing the pharmaceutical formulations of the present invention are according to the standards of ASTM (American Society for Testing Materials).

In some embodiments, the invention includes use of packaging materials such as containers and closures of high-density polyethylene (HDPE), low-density polyethylene (LDPE) and or polypropylene and/or glass, and blisters or strips composed of aluminum or high-density polypropylene, polyvinyl chloride, polyvinylidene dichloride, etc.

In aspects, the invention includes methods of treating patients suffering from pain, inflammation and/or other conditions using pharmaceutical formulations of the present invention. In aspects, the invention includes methods of reducing the risk of gastrointestinal side effects in people taking naproxen for pain relief and for other conditions using pharmaceutical formulations of the present invention.

The pharmaceutical dosage forms of the present invention are intended for oral administration to a patient in need thereof.

Certain specific aspects and embodiments of the invention will be explained in more detail with reference to the following examples, being provided only for purposes of illustration, and it is to be understood that the present invention is not deemed to be limited thereto.

### Example 1

### Enteric Coated Naproxen Tablet, with Naproxen and Esomeprazole Layer Coatings

| | |
|---|---|
| Ingredient | mg/Tablet |

Core Tablet

| | |
|---|---|
| Naproxen | 460 |
| Microcrystalline cellulose | 115 |
| Povidone (PVP K30) | 20 |
| Talc | 15 |
| Magnesium stearate | 10 |
| Tablet Total | 620 |

Enteric Coating

| | |
|---|---|
| Eudragit ™ L-100-55** | 37.2 |
| Triethyl citrate | 3.72 |
| Talc | 18.6 |
| Water* | q.s. |
| Isopropyl alcohol* | q.s. |
| Coating Total | 59.52 |

Naproxen Layer

| | |
|---|---|
| Naproxen | 40 |
| Eudragit L-100-55 | 7 |
| Triethyl citrate | 0.7 |
| Talc | 4.1 |
| Isopropyl alcohol* | q.s. |
| Layer Total | 51.8 |

Esomeprazole Layer

| | |
|---|---|
| Esomeprazole magnesium | 20 |
| Opadry ™ Clear YS-1-7006*** | 15 |
| Water* | q.s. |
| Isopropyl alcohol* | q.s. |
| Layer Total | 35 |

*Evaporates during processing.
**Eudragit L-100-55 is sold by Evonik Industries Ltd., Germany, and contains anionic copolymer based on methacrylic acid and ethyl acrylate.
***Opadry Clear is a product of Colorcon, containing 91% w/w of hydroxypropyl methylcellulose (E-6) and 9% w/w of polyethylene glycol.

Manufacturing Process:

Core Tablet

1. Sift naproxen and microcrystalline cellulose through a #20 mesh sieve and mix.

2. Dissolve povidone in water and use to granulate the dry mixture of step 1.

3. Dry the wet mass at 70° C. for 1 hour and sift the granules through a #30 mesh sieve.

4. Sift talc through a #40 mesh sieve and mix with the dried granules of step 3.

5. Sift magnesium stearate through a #40 mesh sieve and mix with the blend of step 4.

6. Compress the lubricated blend of step 5 into tablets.

7. Disperse Eudragit L-100-55, triethyl citrate and talc in a mixture of water and isopropyl alcohol.

8. Coat the tablets of step 6 with the coating dispersion of step 7.

Naproxen Layer

9. Disperse Eudragit L-100-55, triethyl citrate and talc in isopropyl alcohol.

10. Dissolve naproxen in the dispersion of step 9.

11. Coat the naproxen enteric coated tablets of step 8 with the dispersion of step 10.

Esomeprazole Layer

12. Dissolve esomeprazole magnesium in a dispersion of Opadry Clear in water and isopropyl alcohol.

13. Coat the layered tablets of step 11 with the dispersion of step 12.

### Examples 2-5

### Enteric Coated Naproxen Tablets with Naproxen and Esomeprazole Layers and Optional Barrier Layers

| | mg/Tablet | | | |
|---|---|---|---|---|
| Ingredient | 2 | 3 | 4 | 5 |

Core

| | | | | |
|---|---|---|---|---|
| Naproxen | 460 | 460 | 460 | 460 |
| Microcrystalline cellulose | 115 | 115 | 115 | 115 |
| Povidone (PVP K30) | 20 | 20 | 20 | 20 |
| Talc | 15 | 15 | 15 | 15 |
| Magnesium stearate | 10 | 10 | 10 | 10 |
| Core Total | 620 | 620 | 620 | 620 |

Barrier Layer

| | | | | |
|---|---|---|---|---|
| Opadry Clear YS-1-7006 | 18 | 18 | — | — |
| Water* | q.s. | q.s. | — | — |
| Layer Total | 18 | 18 | — | — |

Enteric Coating

| | | | | |
|---|---|---|---|---|
| Eudragit L-100-55 | 37.2 | 37.2 | 37.2 | 37.2 |
| Triethyl citrate | 3.72 | 3.72 | 3.72 | 3.72 |
| Talc | 18.6 | 18.6 | 18.6 | 18.6 |
| Water* | q.s. | q.s. | q.s. | q.s. |
| Isopropyl alcohol* | q.s. | q.s. | q.s. | q.s. |
| Coating Total | 59.52 | 59.52 | 59.52 | 59.52 |

Naproxen Layer

| | | | | |
|---|---|---|---|---|
| Naproxen | 40 | 40 | 40 | 40 |
| Opadry Clear YS-1-7006 | — | — | 30 | 30 |
| Eudragit L-100-55 | 7 | 7 | — | — |
| Triethyl citrate | 0.7 | 0.7 | — | — |
| Talc | 4.1 | 4.1 | — | — |
| Water* | q.s. | q.s. | q.s. | q.s. |
| Isopropyl alcohol* | q.s. | q.s. | — | — |
| Layer Total | 51.8 | 51.8 | 70 | 70 |

Esomeprazole Layer

| | | | | |
|---|---|---|---|---|
| Esomeprazole magnesium | 20 | 20 | 20 | 20 |
| Opadry Clear YS-1-7006 | 15 | 15 | 15 | 15 |
| Water* | q.s. | q.s. | q.s. | q.s. |
| Isopropyl alcohol* | q.s. | q.s. | q.s. | q.s. |
| Layer Total | 35 | 35 | 35 | 35 |

Barrier Layer

| | | | | |
|---|---|---|---|---|
| Opadry Clear YS-1-7006 | — | 24 | — | 25 |
| Water* | — | q.s. | — | q.s. |
| Layer Total | — | 24 | — | 25 |

*Evaporates during processing.

Manufacturing processes are similar to that of Example 1, with the following differences:

Example 2: a barrier layer is applied between the naproxen core and enteric coating.

Example 3: a barrier layer is applied between the naproxen core and enteric coating, and another barrier layer is applied over the esomeprazole layer.

Example 4: Opadry Clear YS-1-7006 is used in the naproxen layer instead of Eudragit L-100-55.

Example 5: Opadry Clear YS-1-7006 is used in the naproxen layer instead of Eudragit L-100-55, and a barrier layer is applied over the esomeprazole layer.

### Example 6

### Naproxen Enteric Coated Multiparticulates and Esomeprazole Immediate Release Blend, Compressed into a Tablet

| | |
|---|---|
| Ingredient | mg/Tablet |

Naproxen Pellets

| | |
|---|---|
| Naproxen | 500 |
| Microcrystalline cellulose (Avicel ™ PH200) | 64 |
| Povidone (PVP K90) | 6 |
| Pellet Total | 570 |

Enteric Coating (Part I)

| | |
|---|---|
| Eudragit L-100 | 46.74 |
| Diethyl phthalate | 9.69 |
| Acetone | q.s. |
| Isopropyl alcohol | q.s. |
| Part I Total | 56.43 |

Enteric Coating (Part II)

| | |
|---|---|
| Eudragit L-100 | 6.26 |
| Diethyl phthalate | 10.65 |
| Acetone* | q.s. |
| Isopropyl alcohol* | q.s. |
| Part II Total | 16.91 |

Esomeprazole Blend

| | |
|---|---|
| Esomeprazole | 20 |
| Microcrystalline cellulose (Avicel PH101) | 78 |
| Magnesium stearate | 2 |
| Blend Total | 100 |

Compression

| | |
|---|---|
| Microcrystalline cellulose (Avicel PH102) | 300 |
| Lactose | 70 |
| Magnesium stearate | 10 |
| Compression Total | 380 |

*Evaporates during processing.

Manufacturing Process:

Naproxen Pellets

1. Sift naproxen and Avicel PH200 through a #20 mesh sieve and mix.

2. Dissolve povidone in water and use to granulate the dry mixture of step 1.

3. Mix the wet mass of step 2.

4. Pass the wet mass through an extruder-spheronizer to form pellets.

5. Dry the pellets of step 4 and separate into two portions, Part I having 92% of naproxen pellets and Part II having 8% of naproxen pellets, by weight.

Enteric Coating (Part I)

6. Dissolve Eudragit L-100 and diethyl phthalate in a mixture of isopropyl alcohol and acetone.

7. Spray the coating solution of step 6 onto 92% w/w of naproxen pellets of step 5.

Enteric Coating (Part II)

8. Dissolve Eudragit L-100 and diethyl phthalate in a mixture of isopropyl alcohol and acetone.

9. Spray the coating solution of step 8 onto 8% w/w of naproxen pellets of step 5.

Esomeprazole Blend

10. Sift esomeprazole and Avicel PH101 through a #20 mesh sieve and mix.

11. Sift magnesium stearate through a #40 mesh sieve and mix with the blend of step 10.

Compression

12. Sift Avicel PH102 and lactose through a #20 mesh sieve and mix.

13. Mix the blend of step 12 with esomeprazole blend of step 11, enteric coated pellets of step 7, and enteric coated pellets of step 9.

14. Sift magnesium stearate through a #40 mesh sieve and mix with the blend of step 13.

15. Compress the lubricated blend of step 14 into tablets.

### Examples 7-10

### Naproxen Enteric Coated Multiparticulates and Esomeprazole Immediate Release Blend, Compressed into Tablets

| | mg/Tablet | | | |
|---|---|---|---|---|
| Ingredient | 7 | 8 | 9 | 10 |

Naproxen Pellets

| | | | | |
|---|---|---|---|---|
| Naproxen | 500 | 500 | 500 | 500 |
| Microcrystalline cellulose | 64 | 64 | 64 | 64 |
| (Avicel PH200) | | | | |
| Povidone (PVP K90) | 6 | 6 | 6 | 6 |
| Pellet Total | 570 | 570 | 570 | 570 |

Barrier Layer

| | | | | |
|---|---|---|---|---|
| Povidone K30 | 28.5 | 57 | 57 | 57 |
| Alcohol* | q.s. | q.s. | q.s. | q.s. |
| Layer Total | 28.5 | 57 | 57 | 57 |

Enteric Coating (Part I)

| | | | | |
|---|---|---|---|---|
| Eudragit L-100 | 49.8 | 49.8 | 49.8 | 49.8 |
| Diethyl phthalate | 10.17 | 10.17 | 10.17 | 10.17 |
| Acetone* | q.s. | q.s. | q.s. | q.s. |
| Isopropyl alcohol* | q.s. | q.s. | q.s. | q.s. |
| Part I Coating Total | 59.97 | 59.97 | 59.97 | 59.97 |

Enteric Coating (Part II)

| | | | | |
|---|---|---|---|---|
| Eudragit L-100 | 6.5 | 6.5 | 6.5 | 6.5 |
| Diethyl phthalate | 11.05 | 11.05 | 11.05 | 11.05 |
| Acetone* | q.s. | q.s. | q.s. | q.s. |
| Isopropyl alcohol* | q.s. | q.s. | q.s. | q.s. |
| Part II Coating Total | 17.55 | 17.55 | 17.55 | 17.55 |

Esomeprazole Blend

| | | | | |
|---|---|---|---|---|
| Esomeprazole | 20 | 20 | 20 | 20 |
| Sodium bicarbonate | — | — | 140 | 140 |
| Hydroxypropyl | — | 2 | — | 2 |
| methylcellulose (HPMC) | | | | |
| Microcrystalline cellulose | — | 76 | — | 76 |
| (Avicel PH101) | | | | |
| Microcrystalline cellulose | 78 | — | 36 | — |
| (Avicel PH102) | | | | |
| Water* | — | q.s. | — | q.s. |
| Magnesium stearate | 2 | 2 | 4 | 2 |
| Blend Total | 100 | 100 | 200 | 240 |

Compression

| | | | | |
|---|---|---|---|---|
| Microcrystalline cellulose | 300 | 300 | 300 | 300 |
| (Avicel PH102) | | | | |
| Lactose | 70 | 70 | 70 | 70 |
| Magnesium stearate | 10 | 10 | 10 | 10 |
| Compression Total | 380 | 380 | 380 | 380 |

*Evaporates during processing.

Manufacturing processes are similar to that of Example 6, with the following differences:

Example 7: a barrier layer coating is applied to naproxen pellets before enteric coating.

Example 8: a barrier layer coating is applied to naproxen pellets before enteric coating, and there is an additional ingredient HPMC in the esomeprazole blend.

Example 9: a barrier layer coating is applied to naproxen pellets before enteric coating, and there is an additional ingredient sodium bicarbonate in the esomeprazole blend.

Example 10: a barrier layer coating is applied to naproxen pellets before enteric coating, and there are additional ingredients HPMC and sodium bicarbonate in the esomeprazole blend.

### Examples 11-13

### Naproxen Tablet With Permeable Enteric Coating and Esomeprazole Layer

| | mg/Tablet | | |
|---|---|---|---|
| Ingredient | 11 | 12 | 13 |

Core

| | | | |
|---|---|---|---|
| Naproxen | 500 | 500 | 500 |
| Microcrystalline cellulose | 115 | 115 | 115 |
| Povidone (PVP K30) | 20 | 20 | 20 |
| Talc | 15 | 15 | 15 |
| Magnesium stearate | 10 | 10 | 10 |
| Core Total | 660 | 660 | 660 |

Enteric Coating

| | | | |
|---|---|---|---|
| Eudragit L-100-55 | 40 | 40 | 40 |
| Triethyl citrate | 4 | 4 | 4 |
| Lactose | 6 | — | — |
| Span ™ 60** | — | 4 | — |
| Tween ™ 80*** | — | — | 2 |
| Talc | 20 | 20 | 20 |
| Water* | q.s. | q.s. | q.s. |
| Isopropyl alcohol* | q.s. | q.s. | q.s. |
| Layer Total | 70 | 68 | 66 |

Esomeprazole Layer

| | | | |
|---|---|---|---|
| Esomeprazole magnesium | 20 | 20 | 20 |
| Opadry Clear YS-1-7006 | 15 | 15 | 15 |
| Water* | q.s. | q.s. | q.s. |
| Isopropyl alcohol* | q.s. | q.s. | q.s. |
| Layer Total | 35 | 35 | 35 |

*Evaporates during processing.
**SPAN 60 is Sorbitan monostearate.
***Span 80 is Sorbitan monooleate.

Example 11 Manufacturing Process:

Core

1. Sift naproxen and microcrystalline cellulose through a #20 mesh sieve and mix.

2. Dissolve povidone in water and use to granulate the dry mixture of step 1.

3. Dry the wet mass at 70° C. for 1 hour and sift the granules through a #30 mesh sieve.

4. Sift talc through a #40 mesh sieve and mix with the dried granules of step 3.

5. Sift magnesium stearate through a #40 mesh sieve and mix with the blend of step 4.

6. Compress the lubricated blend of step 5 into tablets.

Enteric Coating

7. Disperse Eudragit L-100-55, triethyl citrate, lactose and talc in a mixture of water and isopropyl alcohol.

8. Coat the tablets of step 6 with the dispersion of step 7.

Esomeprazole Layer

9. Dissolve esomeprazole magnesium in a dispersion of Opadry Clear in water and isopropyl alcohol.

10. Coat the layered tablets of step 8 with the dispersion of step 9.

Example 12 manufacturing process is similar to that of Example 11, except that Span 60 is used instead of lactose in the enteric coating.

Example 13 manufacturing process is similar to that of Example 11, except that Tween 80 is used instead of lactose in the enteric coating.

### Examples 14-15

### Naproxen Tablets with Esomeprazole Coating

mg/Tablet

| | | |
|---|---|---|
| Ingredient | 14 | 15 |

Core

| | | |
|---|---|---|
| Naproxen | 500 | 500 |
| Povidone (PVP K90) | — | 15 |
| Eudragit L30 D55 | 50 | 45 |
| Water* | q.s. | qs |
| Lactose monohydrate | 15 | — |
| Talc | | 13.5 |
| Croscarmellose sodium | 10 | 14.4 |
| Magnesium stearate | 3 | 3 |
| Core Total | 578 | 590.9 |

Subcoating

| | | |
|---|---|---|
| Opadry Clear 39B590000 | 28.9 | 29.5 |
| Water* | q.s. | q.s. |
| Coating Total | 28.9 | 29.5 |

Drug Layering

| | | |
|---|---|---|
| Esomeprazole magnesium | 20.7 | 20.71 |
| Hypromellose 3 cP | 10 | 10 |
| Povidone (PVP K30) | 5 | 5 |
| Macrogol PEG 400 | 4 | 4 |
| Poloxamer 188 (Lutrol ™ F68) | 3 | 3 |
| Croscarmellose sodium | 4 | 4 |
| Lactose monohydrate | 23 | 23 |
| Magnesium oxide light | 5 | 5 |
| Methanol* | q.s. | q.s. |
| Water* | q.s. | q.s. |
| Drug Layer Total | 74.7 | 74.7 |

Protective Coating

| | | |
|---|---|---|
| Hypromellose 3 cP | 3.5 | 3.5 |
| Lactose monohydrate | 10.5 | 10.5 |
| Croscarmellose sodium | 4.8 | 4.8 |
| Macrogol PEG 400 | 0.44 | 0.4 |
| Iron oxide, yellow | 0.12 | 0.12 |
| Water* | q.s. | q.s. |
| Coating Total | 19.4 | 19.4 |
| Tablet Total | 701.01 | 714.5 |

*Evaporates during processing

Manufacturing process for Example 14:

1. Naproxen is granulated with a 10% weight by weight dispersion of Eudragit L30-D55 in water loaded in a fluid bed coater, using a top spray technique. The granules are dried.

2. Lactose and croscarmellose sodium are added to the granules and blended for about 5 minutes.

3. Magnesium stearate is added and blended for about 5 minutes, then the mixture is compressed into tablets.

4. Tablets are coated with an Opadry dispersion using a coating pan, to produce a 5% weight gain, and then are dried.

5. Drug layering ingredients are mixed to form dispersion and sprayed on to the sub-coated tablets by pan coating.

6. Drug layered tablets are coated with a dispersion of protective coating ingredients, and dried.

Manufacturing Process for Example 15:

1. Naproxen is granulated with a povidone solution by top spray in a fluid bed coater, and the granules are dried.

2. Dry granules are coated with a 10% w/w dispersion of Eudragit L30-D55 in water by top spray, and the granules are dried to a loss on drying (LOD) of 2% or less.

3. Croscarmellose sodium is blended with the granules for 5 minutes.

4. Magnesium stearate is added and blended for 5 minutes.

5. The mixture is compressed into tablets.

6. Tablets are coated with Opadry dispersion in a coating pan, to produce at 5% weight gain, after drying.

7. Drug layering ingredients are mixed to form dispersion and sprayed on to the sub-coated tablets by pan coating.

8. Drug layered tablets are coated with a dispersion of protective coating ingredients, and dried.

### Example 16

### Esomeprazole and Naproxen Tri-Layer Tablets

| | |
|---|---|
| Ingredient | mg/Tablet |

Naproxen Layer (Layer 1)

| | |
|---|---|
| Naproxen | 500 |
| Eudragit L30 D55 | 50 |
| Water* | q.s. |
| Lactose monohydrate | 15 |
| Croscarmellose sodium | 10 |
| Magnesium stearate | 3 |
| Layer Total | 578 |

Intermediate Layer (Layer 2)

| | |
|---|---|
| Microcrystalline cellulose | 10 |
| Lactose monohydrate | 100 |
| Povidone (PVP K90) | 20 |
| Magnesium stearate | 5 |
| Layer Total | 225 |

Drug Layer (Layer 3)

| | |
|---|---|
| Esomeprazole magnesium | 20.7 |
| Povidone (PVP K30) | 20 |
| Microcrystalline cellulose | 100 |
| Croscarmellose sodium | 15 |
| Meglumine | 5 |
| Methanol* | q.s. |
| Croscarmellose sodium | 10 |
| Microcrystalline cellulose | 150 |
| Magnesium stearate | 5.00 |
| Layer Total | 325.7 |

Final Coating

| | |
|---|---|
| Opadry Yellow | 33.8 |
| Water* | q.s. |
| Coating Total | 33.8 |
| Tablet Total | 1162.5 |

*Evaporates during processing.

Manufacturing Process:

I) Layer 1

1. Naproxen is granulated with a 10% w/w dispersion of Eudragit L30-D55 in a fluid bed processor, with top spray. The granules are dried to a LOD less than 2% w/w.

2. Lactose and croscarmellose sodium are added and blended for 5 minutes.

3. Magnesium stearate was added and blended for 5 minutes. II) Layer 2 1. Microcrystalline cellulose, lactose and povidone are blended for 5 minutes.

2. Magnesium stearate is added and blended for 5 minutes. III) Layer 3

1. Esomeprazole magnesium, povidone, microcrystalline cellulose, and croscarmellose sodium ares mixed in a rapid mixer granulator for 5 minutes.

2. Meglumine is dissolved in methanol and used to granulate the blend of step 1. Granules are dried in a fluid bed dryer to a LOD less than 2%.

3. Granules are blended with croscarmellose sodium and microcrystalline cellulose for 5 minutes.

4. Magnesium stearate is added and blended for 5 minutes. IV) Tableting

1. Layer 1 is placed into a die and lightly compressed. Layer 2 is added to the die and lightly compressed. Layer 3 is added and compressed to form a tri-layer tablet.

2. Tablets are coated with the protective coating dispersion by pan coating, to produce a 3% weight gain, after drying.

### Example 17

### Esomeprazole and Naproxen Tablets with Naproxen in Enteric Coating

| | |
|---|---|
| Ingredient | mg/Tablet |

Core

| | |
|---|---|
| Naproxen | 465 |
| Povidone (PVP K90) | 15 |
| Croscarmellose sodium | 15 |
| Water* | q.s. |
| Croscarmellose sodium | 15 |
| Magnesium stearate | 3 |

Enteric Coating

| | |
|---|---|
| Naproxen | 35 |
| Eudragit ™ L30 D55** | 45 |
| Talc | 9 |
| Triethyl citrate | 4.5 |
| Sodium hydroxide | 1.13 |
| Water* | q.s. |
| Coating Total | 94.63 |

Subcoating

| | |
|---|---|
| Opadry Clear 39B590000 | 25.65 |
| Water* | q.s. |
| Coating Total | 25.65 |

Drug Layering

| | |
|---|---|
| Esomeprazole magnesium | 20.71 |
| Hypromellose 3 cP | 10 |
| Povidone (PVP K30) | 5 |
| Macrogol PEG 400 | 4 |
| Poloxamer 188 (Lutrol F68) | 3 |
| Croscarmellose sodium | 4 |
| Lactose monohydrate | 23 |
| Magnesium oxide light | 5 |
| Methanol* | q.s. |
| Water* | q.s. |
| Drug Layer Total | 74.71 |

Protective Coating

| | |
|---|---|
| Hypromellose 3 cP | 3.49 |
| Lactose monohydrate | 10.55 |
| Croscarmellose sodium | 4.80 |
| Macrogol PEG 400 | 0.44 |
| Iron oxide, yellow | 0.12 |
| Water* | q.s. |
| Coating Total | 19.4 |

*Evaporates during processing.
**Eudragit L30 D55 is a product of Evonik Industries Ltd, Germany, and contains anionic copolymer based on methacrylic acid and ethyl acrylate.

Manufacturing Process:

1. Naproxen and croscarmellose sodium (first quantity) are mixed and granulated using a solution of povidone and water, in a fluid bed processor with top spray. The granules are dried to a LOD less than 2% w/w.

2. Croscarmellose sodium (second quantity) is added and blended for 5 minutes.

3. Magnesium stearate is added and blended for 5 minutes.

4. The blend is compressed into tablets.

5. Talc, triethyl citrate, and sodium hydroxide are combined with Eudragit L30-D55, the mixture is stirred for 30 minutes, then naproxen is added and stirring is continued for 15 minutes.

6. The dispersion is coated onto tablets, to produce an 18.5% weight gain, after drying.

7. Tablets are coated with subcoating ingredients.

8. Drug loading ingredients are mixed & made dispersion and used to coat the subcoated tablets by pan coating.

9. Drug layered tablets are coated with a mixture of protective coating ingredients.

### Example 18

### Naproxen and Esomeprazole Tablets

| | |
|---|---|
| Ingredient | mg/Tablet |

Core

| | |
|---|---|
| Naproxen | 465 |
| Povidone (PVP K90) | 15 |
| Croscarmellose sodium | 15 |
| Water* | q.s. |
| Croscarmellose sodium | 15 |
| Magnesium stearate | 3 |
| Core Total | 513 |

Enteric Coating (Part 1)

| | |
|---|---|
| Eudragit L30 D55 | 35 |
| Talc | 7 |
| Triethyl citrate | 3.5 |
| Sodium hydroxide | 0.88 |
| Water* | q.s. |
| Coating Total | 46.38 |

Enteric Coating (Part 2)

| | |
|---|---|
| Naproxen | 35 |
| Eudragit L30 D55 | 15 |
| Talc | 3 |
| Triethyl citrate | 1.5 |
| Sodium hydroxide | 0.38 |
| Povidone (PVP K30) | 15 |
| Water* | q.s. |
| Coating Total | 69.88 |

Subcoating

| | |
|---|---|
| Opadry Clear 39B590000 | 25.65 |
| Water* | q.s. |
| Coating Total | 25.65 |

Drug Layering

| | |
|---|---|
| Esomeprazole magnesium | 20.71 |
| Hypromellose 3 cP | 10 |
| Povidone (PVP K30) | 5 |
| Macrogol PEG 400 | 4 |
| Poloxamer 188 (Lutrol F68) | 3 |
| Croscarmellose sodium | 4 |
| Lactose monohydrate | 23 |
| Magnesium oxide light | 5 |
| Methanol* | q.s. |
| Water* | q.s. |
| Drug Layer Total | 74.71 |

Protective Coating

| | |
|---|---|
| Hypromellose 3 cP | 3.49 |
| Lactose monohydrate | 10.55 |
| Croscarmellose sodium | 4.8 |
| Macrogol PEG 400 | 0.44 |
| Iron oxide, yellow | 0.12 |
| Water* | q.s. |
| Coating Total | 19.4 |
| Tablet Total | 632.76 |

*Evaporates during processing.

Manufacturing Process:

Core:

1. Naproxen and croscarmellose sodium (first quantity) are mixed and granulated with a solution of povidone in water, in a fluid bed processor with top spray. Granules are dried to a LOD less than 2% w/w.

2. Croscarmellose sodium (second quantity) is added and blended for 5 minutes.

3. Magnesium stearate is added and blended for 5 minutes.

4. The mixture is compressed into tablets.

Enteric Coating (Part 1):

5. Talc, triethyl citrate, and sodium hydroxide are combined with Eudragit L30-D55 and the mixture is stirred for 30 minutes.

6. The dispersion is coated onto tablets to produce a 7.3% weight gain, after drying.

Enteric Coating (Part 2):

7. Talc, triethyl citrate, and sodium hydroxide are combined with Eudragit L30-D55 and the mixture is stirred for 30 minutes and naproxen is added and stirring is continued for 15 minutes.

8. The dispersion is coated onto tablets to produce an 11.05% weight gain, after drying.

9. Subcoating ingredients are mixed and coated onto the tablets.

10. Drug loading ingredients are mixed to make dispersion and coated onto subcoated tablets by pan coating.

11. Drug layered tablets are coated with a mixture of the protective coating ingredients.

### Example 19

### Naproxen and Esomeprazole Tablets

| | |
|---|---|
| Ingredient | mg/Tablet |

Core

| | |
|---|---|
| Naproxen | 500 |
| Povidone (PVP K90) | 15 |
| Croscarmellose sodium | 15 |
| Water* | q.s. |
| Croscarmellose sodium | 15 |
| Magnesium stearate | 3 |
| Core Total | 548 |

Delayed Release Coating

| | |
|---|---|
| Ethylcellulose | 35 |
| HPMC K15M Premium CR | 7 |
| Triethyl citrate | 4.2 |
| Isopropyl alcohol* | q.s. |
| Methylene chloride* | q.s. |
| Coating Total | 46.2 |

Subcoating

| | |
|---|---|
| Opadry Clear 39B590000 | 27.4 |
| Water* | q.s. |
| Coating Total | 27.4 |

Drug Layering

| | |
|---|---|
| Esomeprazole magnesium | 20.71 |
| Hypromellose 3 cP | 10 |
| Povidone (PVP K30) | 5 |
| Macrogol PEG 400 | 4 |
| Poloxamer 188 (Lutrol F68) | 3 |
| Croscarmellose sodium | 4 |
| Lactose monohydrate | 23 |
| Magnesium oxide light | 5 |
| Methanol* | q.s. |
| Water* | q.s. |
| Drug Layer Total | 74.71 |

Protective Coating

| | |
|---|---|
| Opadry Brown 02B86964 | 20.9 |
| Water* | q.s. |
| Coating Total | 20.9 |
| Tablet Total | 671.01 |

*Evaporates during processing.

Manufacturing Process:

1. Naproxen and croscarmellose sodium (first quantity) are mixed and granulated with a solution of povidone in water, in a fluid bed processor with top spray. Granules are dried to a LOD less than 2% w/w.

2. Croscarmellose sodium (second quantity) is added and blended for 5 minutes.

3. Magnesium stearate is added and blended for 5 minutes.

4. The mixture is compressed into tablets.

5. Ethyl cellulose, HPMC, and triethyl citrate are dispersed in a mixture of isopropyl alcohol and methylene chloride (1:1 volume ratio).

6. The dispersion is coated onto tablets to produce a 8-9% weight gain, after drying.

7. Subcoating ingredients are mixed to form a suspension and coated onto the tablets, then the tablets are dried.

8. Drug loading ingredients are mixed to form a dispersion and coated onto subcoated tablets by pan coating, then the tablets are dried.

9. Drug layered tablets are coated with a mixture of the protective coating ingredients, and dried.

### Example 20

### Drug Dissolution

Tablets prepared in Examples 14-19 are tested for naproxen component release using USP apparatus 2 (paddle) and 1000 mL of pH 3.5 citrate buffer, with 50 RPM stirring. The results are shown in Table 1.

TABLE 1

Cumulative % of Drug Released

| Minutes | 14 | 15 | 16 | 17 | 18 | 19 |
|---|---|---|---|---|---|---|
| 5 | 0 | 0 | 0 | 0 | 0 | — |
| 10 | 0 | 0 | 1 | 0 | 0 | 0 |
| 15 | 1 | 0 | 2 | 1 | 0 | — |
| 30 | 3 | 2 | 6 | 4 | 2 | 0 |
| 45 | 8 | 7 | 9 | 8 | 9 | — |
| 60 | 12 | 12 | 13 | 12 | 14 | 1 |

Tablets prepared in Examples 14-19 for acid resistance were tested for naproxen component release in USP apparatus 2 (paddle), using 1000 mL of 0.1N HCl for the first two hours, then 1000 mL of pH 6.8 phosphate buffer, with 50 rpm stirring. Results are shown in Table 2.

TABLE 2

Cumulative % of Drug Released

| Time | 14 | 15 | 16 | 17 | 18 | 19 |
|---|---|---|---|---|---|---|
| 2 hours | 0 | 0 | 0 | 0 | 0 | 0 |
| 2 hours, 45 minutes | 92 | 100 | 88 | 92 | 97 | 87 |

Tablets prepared in Examples 14-19 are tested for esomeprazole component release using USP apparatus 1 (basket), 1000 mL of pH 6.8 phosphate buffer, and 50 rpm rotation. The results are shown in Table 3.

TABLE 3

Cumulative % of Drug Released

| Minutes | 14 | 15 | 16 | 17 | 18 | 19 |
|---|---|---|---|---|---|---|
| 5 | 2 | 16 | 2 | 14 | 5 | 2 |
| 10 | 7 | 34 | 11 | 25 | 14 | 11 |
| 15 | 16 | 54 | 27 | 25 | 26 | 27 |
| 30 | 52 | 92 | 75 | 63 | 58 | 75 |
| 45 | 83 | 95 | 96 | 86 | 74 | 96 |
| 60 | 85 | 94 | 96 | 101 | 93 | 96 |

We claim:

1. A pharmaceutical fixed unit dosage formulation for oral administration comprising a proton pump inhibitor and a non-steroidal anti-inflammatory drug, wherein at least a portion of non-steroidal anti-inflammatory drug is released when the formulations are immersed into aqueous fluids having pH values below about 3.5.

2. A pharmaceutical fixed unit dosage formulation for oral administration comprising a non-steroidal anti-inflammatory drug and a proton pump inhibitor, wherein a portion of a non-steroidal anti-inflammatory drug is released when the formulation is immersed in aqueous media having pH values less than 3.5, and a remaining portion of a non-steroidal anti-inflammatory drug is released when the formulation is immersed in aqueous media having pH values 3.5 or higher.

3. A pharmaceutical fixed unit dosage formulation for oral administration comprising a non-steroidal anti-inflammatory drug and a proton pump inhibitor, wherein at least a portion of a non-steroidal anti-inflammatory drug and at least a portion of proton pump inhibitor are released when the formulation is immersed in aqueous media having pH values less than about 3.5.

4. A pharmaceutical fixed unit dosage formulation for oral administration comprising a non-steroidal anti-inflammatory drug and a proton pump inhibitor, wherein a portion of a non-steroidal anti-inflammatory drug and at least a portion of proton pump inhibitor are released when the formulation is immersed in aqueous media having pH values less than 3.5, and a remaining portion of a non-steroidal anti-inflammatory drug is released when the formulation is immersed in aqueous media having pH values 3.5 or higher.

5. The pharmaceutical formulation of any of claims 1-4, wherein a non-steroidal anti-inflammatory drug comprises acetylsalicylic acid or naproxen.

6. The pharmaceutical formulation of any of claims 1-4, wherein a proton pump inhibitor comprises omeprazole, esomeprazole, lansoprazole, dexlansoprazole, pantaprozole, tanataprazole, or rabeprazole.

7. The pharmaceutical formulation of any of claims 1-4, wherein a non-steroidal anti-inflammatory drug is present in amounts between about 50 mg and about 1500 mg.

8. The pharmaceutical formulation of any of claims 1-4, wherein a proton pump inhibitor is present in amounts between about 10 mg and about 100 mg.

9. A method of relieving signs and symptoms of any of osteoarthritis, rheumatoid arthritis, and ankylosing spondylitis, comprising administering a pharmaceutical formulation of any of claims 1-4.

10. A method of treating non-steroidal anti-inflammatory drug-associated gastric ulcers, comprising administering a pharmaceutical formulation of any of claims 1-4.
